(19) United States
(12) Patent Application Publication
(10) Pub. No.: US 2015/0202020 A1
(43) Pub. Date: Jul. 23, 2015

(54) METHOD FOR EPICARDIAL PACING OR CARDIAC TISSUE ABLATION

(71) Applicant: MONTEFIORE MEDICAL CENTER, Bronx, NY (US)

(72) Inventor: John Devens Fisher, Stamford, CT (US)

(21) Appl. No.: 14/416,159

(22) PCT Filed: Jul. 24, 2013
(86) PCT No.: PCT/US13/51797

§ 371 Date: Jan. 21, 2015

Related U.S. Application Data
(60) Provisional application No. 61/674,973, filed on Jul. 24, 2012.

(51) Int. Cl. A61B 19/00; A61B 8/08; A61B 8/02; A61B 18/02; A61N 1/362; A61N 1/05; A61B 8/06; A61B 18/14

(52) U.S. Cl. CPC A61B 19/5244; A61B 8/06; A61B 8/488; A61B 18/1492; A61B 18/02; A61N 1/362; A61N 1/0587; A61B 8/02; A61B 2018/0212; A61B 2018/00363

(57) ABSTRACT

A method is provided for placing an epicardial pacing lead onto the epicardium of a heart in a subject. A method is also provided for placing an ablation catheter onto the epicardium of a heart in a subject. Articles of manufacture or machines for use in the methods are also provided.

### CROSS-REFERENCE TO RELATED APPLICATIONS

This application claims benefit of U.S. Provisional Application No. 61/674,973,filed Jul. 24, 2012, the contents of which are hereby incorporated by reference.

### BACKGROUND OF THE INVENTION

Throughout this application various publications, patents, patent application publications and books are referred to. Full citations for the publications may be found at the end of the specification. The disclosures of the publications, patents, patent application publications and books are hereby incorporated by reference in their entirety into the subject application to more fully describe the art to which the subject invention pertains.

In patients with heart failure, the electrical impulses that cause squeezing or contraction, and therefore pumping by the lateral part of the left ventricle, are often slower than normal. This results in an asymmetrical or desynchronized left ventricular contraction that compromises the pumping ability of an already weakened left ventricle. Over the last 10-15 years, several techniques have been developed for cardiac resynchronization therapy (CRT). To accomplish CRT, one pacing electrode is placed in the right ventricle, which shares its septal wall with the left ventricle. An additional pacing electrode is then placed on the free wall of the left ventricle. When both pacing electrodes are stimulated simultaneously, this biventricular (Bi-V) pacing results in CRT. Left ventricular or other epicardial pacing may also be desirable in patients with poor vascular access or other reasons to avoid transvenous lead placement.

The major challenge has been placement of the left ventricular lead. For this, there are three major approaches:
1. Coronary sinus (CS) approach. The CS is a large vein that can be accessed from the right atrium, and which then wraps around the base of the left ventricle with branches spreading onto the surface of the left ventricle. If a pacing electrode can be put in one of these branches, it can be used for CRT. Unfortunately, access to the coronary sinus is sometimes difficult, and many patients do not have a suitable vein branch derived from the coronary sinus. Suboptimal locations must sometimes be accepted because of the anatomy of these coronary vein branches, which differ markedly among individuals.
2. Thoracic surgery approach. Using approaches that can range from minimally invasive to major thoracotomy, the surgeon is able to visualize the surface of the left ventricle, and place a pacing electrode/lead in a suitable position. This procedure is usually undertaken when the coronary sinus approach has proved unsuccessful and, therefore, subjects the patient to prolonged hospital stays, further surgery, and risk of infection.
3. Endocardial approach to the left ventricle. A pacing lead is delivered to the inside (endocardial surface) of the left ventricle by any of several routes. Present leads are prone to develop thrombus formation, and these thrombi can break off and embolize, causing strokes and other complications. Thus, the endocardial approach is still highly investigational.

The present invention addresses the need of providing improved techniques for placement of leads, such as the left ventricular lead for epicardial pacing, or of ablation catheters.

### SUMMARY OF THE INVENTION

A method for placing an epicardial pacing lead onto the epicardium of a heart in a subject, comprising placing an end of an epicardial pacing lead delivery sheath, the sheath comprising a lumen and comprising a Doppler blood-flow probe at the end of the delivery sheath, at a predetermined position adjacent to the epicardium of the heart, obtaining a signal from the Doppler blood-flow probe so as to determine the proximity of the end of the sheath to a blood vessel of a predetermined size or above and, when no blood vessel of the predetermined size or above is determined to be within an unacceptable distance of the end of the sheath, advancing an epicardial pacing lead within the lumen of the delivery sheath to the end of the sheath so as to place the epicardial pacing lead onto the epicardium of the heart in the subject.

A method for placing an ablation catheter onto the epicardium of a heart in a subject, comprising placing an end of an ablation catheter delivery sheath, the sheath comprising a lumen and comprising a Doppler blood-flow probe at the end of the delivery sheath, at a predetermined position adjacent to the epicardium of the heart, obtaining a signal from the Doppler blood-flow probe so as to determine the proximity of the end of the sheath to a blood vessel of a predetermined size or above and, when no blood vessel of the predetermined size or above is determined to be within an unacceptable distance of the end of the sheath, advancing an ablation catheter within the lumen of the delivery sheath to the end of the sheath so as to place the ablation catheter onto the epicardium of the heart in the subject.

A method for placing an ablation catheter onto the epicardium of a heart in a subject, comprising placing an end of an ablation catheter delivery sheath, the sheath comprising a lumen, at a first predetermined position adjacent to the epicardium of the heart, advancing an ablation catheter comprising a Doppler blood-flow probe at an end thereof within the lumen of the sheath to the end of the sheath and obtaining a signal from the Doppler blood-flow probe so as to determine the proximity of the end of the ablation catheter to a blood vessel of a predetermined size or above and, when no blood vessel of the predetermined size or above is determined to be within an unacceptable distance of the end of the delivery sheath, placing the ablation catheter comprising the Doppler blood-flow probe onto the epicardium of the heart in the subject.

A method is also provided for placing a pericardiocentesis drainage catheter into the pericardiac space of a heart in a subject, comprising placing an end of a pericardiocentesis needle, the needle comprising a lumen and comprising a Doppler blood-flow probe at the end of the needle, at a predetermined position in the percardiac space of the heart, obtaining a signal from the Doppler blood-flow probe so as to determine the proximity of the end of the needle to a blood vessel of a predetermined size or above and, when no blood vessel of the predetermined size or above is determined to be within an unacceptable distance of the end of the needle, advancing a pericardiocentesis drainage catheter within the lumen of the needle to the end of the needle, or alternatively over a wire guide which has been inserted into the needle and advanced to the tip thereof and subsequently withdrawing the needle, so as to place the pericardiocentesis drainage catheter into the pericardiac space of the heart in the subject.

Also provided is an apparatus comprising an epicardial pacing lead delivery sheath, or an ablation catheter delivery sheath, the delivery sheath comprising a lumen of a diameter sufficient for the advancement of an epicardial pacing lead or an ablation catheter therein, respectively, and comprising a Doppler blood-flow probe at the end of the delivery sheath.

Also provided is a system comprising an apparatus as described herein and a monitor attachable to or attached to the apparatus which monitor displays or emits a signal obtained from the Doppler blood-flow probe.

Additional objects of the invention will be apparent from the description which follows.

### BRIEF DESCRIPTION OF THE FIGURES

FIG. 1: Shows an example of a type of steerable delivery sheath that can be employed with a Doppler probe attached thereto. In this non-limiting example a Medtronic SelectSite® Steerable sheath is shown, designed for delivery of a Medtronic SelectSecure™ pacing lead. A sensor such as a 1 mm Doppler probe with its flexible wire connection (not shown) is bonded to the exterior or interior of the sheath at the delivering end thereof and connected to a measuring device. Such a sheath is compatible with 5F RF Medtronic Marinr® ablation catheters (larger ablation catheters would require a larger sheath).

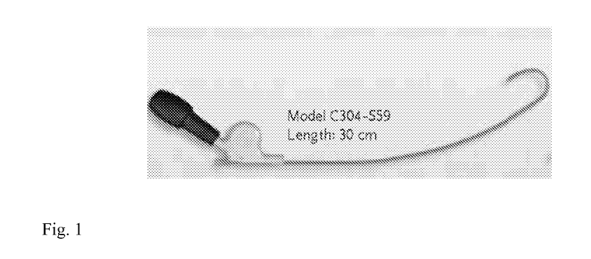

### DETAILED DESCRIPTION OF THE INVENTION

A method for placing an epicardial pacing lead onto the epicardium of a heart in a subject, comprising placing an end of an epicardial pacing lead delivery sheath, the delivery sheath comprising a lumen and comprising a Doppler blood-flow probe at the end of the delivery sheath, at a predetermined position adjacent to the epicardium of the heart, obtaining a signal from the Doppler blood-flow probe so as to determine the proximity of the end of the sheath to a blood vessel of a predetermined size or above and, when no blood vessel of the predetermined size or above is determined to be within an unacceptable distance of the end of the sheath, advancing an epicardial pacing lead within the lumen of the delivery sheath to the end of the sheath so as to place the epicardial pacing lead onto the epicardium of the heart in the subject. In an embodiment, the lead is placed into the pericardial space.

A method for placing an epicardial pacing lead onto the epicardium of a heart in a subject, comprising placing an end of an epicardial pacing lead delivery sheath at a predetermined position adjacent to the epicardium of the heart, the delivery sheath comprising a lumen, advancing an epicardial pacing lead comprising a Doppler blood-flow probe at the tip or end thereof within the lumen of the sheath to the end of the sheath, obtaining a signal from the Doppler blood-flow probe so as to determine the proximity of the end of the sheath to a blood vessel of a predetermined size or above and, when no blood vessel of the predetermined size or above is determined to be within an unacceptable distance of the end of the sheath, advancing the epicardial pacing lead within the lumen of the delivery sheath to the end of the sheath and onto the epicardium of the heart in the subject. In an embodiment, the lead is placed into the pericardial space.

In an embodiment, the methods further comprise administering to the subject an electrical current through the epicardial pacing lead, and through a second epicardial pacing lead positioned in a right ventricle of, or right ventricle portion of a septal wall of, the heart of the subject, so as to deliver a synchronizing electrical current to the heart of the subject.

The lead or leads can be placed passively onto the heart tissue, or can, independently, be attached, for example by a screw attachment mechanism as commonly used in the art. The screw attachment mechanism may be attached to the delivery sheath in an embodiment of the methods and of the apparatuses described herein. In an embodiment the screw attachment mechanism is attached to the end of the delivery sheath placed in close proximity to the cardiac surface or epicardial surface or pericardiac surface.

A method for placing an ablation catheter onto the epicardium of a heart in a subject, comprising placing an end of an ablation catheter delivery sheath, the delivery sheath comprising a lumen and comprising a Doppler blood-flow probe at the end of the delivery sheath, at a predetermined position adjacent to the epicardium of the heart, obtaining a signal from the Doppler blood-flow probe so as to determine the proximity of the end of the sheath to a blood vessel of a predetermined size or above and, when no blood vessel of the predetermined size or above is determined to be within an unacceptable distance of the end of the sheath, advancing an ablation catheter within the lumen of the delivery sheath to the end of the sheath so as to place the ablation catheter onto the epicardium of the heart in the subject. In an embodiment, the lead is placed into the pericardial space.

Also provided is a method for placing an ablation catheter onto the epicardium of a heart in a subject, comprising placing an end of an ablation catheter delivery sheath, the delivery sheath comprising a lumen, at a first predetermined position adjacent to the epicardium of the heart, advancing an ablation catheter comprising a Doppler blood-flow probe at an end thereof within the lumen of the sheath to the end of the sheath and obtaining a signal from the Doppler blood-flow probe so as to determine the proximity of the end of the ablation catheter to a blood vessel of a predetermined size or above and, when no blood vessel of the predetermined size or above is determined to be within an unacceptable distance of the end of the sheath, placing the ablation catheter comprising the Doppler blood-flow probe onto the epicardium of the heart in the subject.

Also provided is a method for placing an epicardial pacing lead onto the epicardium of a heart in a subject, comprising placing an end of an epicardial pacing lead delivery sheath, the delivery sheath comprising a lumen, at a first predetermined position adjacent to the epicardium of the heart, advancing an epicardial pacing lead comprising a Doppler blood-flow probe at an end thereof within the lumen of the sheath to the end of the sheath and obtaining a signal from the Doppler blood-flow probe so as to determine the proximity of the end of the an epicardial pacing lead to a blood vessel of a predetermined size or above and, when no blood vessel of the predetermined size or above is determined to be within an unacceptable distance of the end of the sheath, placing the an epicardial pacing lead comprising the Doppler blood-flow probe onto the epicardium of the heart in the subject.

In an embodiment of the methods for placing an ablation catheter, the method further comprises administering to the subject an electrical current or a radiofrequency energy or a cryogenic material through the ablation catheter to the epicardium in an amount effective to ablate a portion of the cardiac tissue of the heart.

A method is also provided for placing a pericardiocentesis drainage catheter into the pericardiac space of a heart in a subject, comprising placing an end of a pericardiocentesis needle, the needle comprising a lumen and comprising a Doppler blood-flow probe at the end of the needle, at a predetermined position in the percardiac space of the heart, obtaining a signal from the Doppler blood-flow probe so as to determine the proximity of the end of the needle to a blood vessel of a predetermined size or above and, when no blood vessel of the predetermined size or above is determined to be within an unacceptable distance of the end of the needle, advancing a pericardiocentesis drainage catheter within the lumen of the needle to the end of the needle, or alternatively over a wire guide which has been inserted into the needle and advanced to the tip thereof and subsequently withdrawing the needle, so as to place the pericardiocentesis drainage catheter into the pericardiac space of the heart in the subject. In an embodiment, the pericardiocentesis needle is 15 G, 16 G, 17 G, 18 G, 19 G or 20 G. In an embodiment, the method further comprises draining fluid from the pericardiac space through the pericardiocentesis drainage catheter.

In an embodiment of the methods described herein, the subject has an arrhythmia. In an embodiment of the methods described herein, the subject has a post-operative arrhythmia. In an embodiment of the methods described herein, the arrhythmia comprises one or more of sinus node dysfunction, junctional ectopic tachycardia, a supraventricular tachycardia or other bradycardias or tachycardias amenable to pacing or ablative therapy, or atrioventricular block. In an embodiment of the methods described herein, the arrhythmia comprises a supraventricular tachycardia and is an atrio-ventrical nodal reentry, atrio-ventricular reentry, atrial flutter or sinus node reentry tachycardia. In an embodiment of the methods described herein, the subject has had cardiac surgery.

In an embodiment of the methods described herein, the epicardial pacing lead delivery sheath, or the ablation catheter delivery sheath, is introduced into the subject via a subxyphoid route. In a preferred embodiment, the subxyphoid route via needle. In an embodiment of the methods the method does not require or does not comprise introduction of a catheter or sheath or lead via a mini-thoracotomy. In an embodiment of the methods the method does not require or does not comprise introduction of a catheter or sheath or lead via a thorascopy. In an embodiment of the methods the method does not require or does not comprise introduction of a catheter or sheath or lead via a robotic or a da Vinci surgery technique.

The Doppler blood-flow probe at the end of the delivery sheath in the methods and apparatuses described herein is attached to the delivery sheath. It is attached such that it is connected to a conductive element which conducts the signal externally to the subject's body, or permits the signal to be conducted externally to the subject's body. The Doppler signal can be monitored externally. The conductor element may be fastened within, or present within, a lumen of the delivery sheath, for example within the lumen of the sheath that can carry the ablation catheter, or that can carry the epicardial pacing lead. In an embodiment, the conductive element is not within the lumen of the sheath that can carry the ablation catheter, or that can carry the epicardial pacing lead. The conductor element may be fastened within, or present within, a second, separate, lumen of the delivery sheath through which the ablation catheter or epicardial pacing lead does not pass. The conductor element may be fastened to an external surface of the delivery sheath, for example, within an insulator. The Doppler probe can thus be attached directly to the end of the delivery sheath or attached through a conductive element to the end of the delivery sheath.

In an embodiment of the methods and apparatuses described herein, the Doppler probe comprises a piezoelectric crystal. In an embodiment of the methods and devices described herein described herein, the Doppler probe is a 20 MHz microvascular implantable Doppler probe.

The Doppler probe in the methods and apparatuses described herein is not on a pacing lead or an ablation catheter. The Doppler probe is at the end of the delivery sheath (the end which is placed on or in proximity to the heart). Doppler probes which could be used are known in the art. U.S. Pat. No. 4,771,788, hereby incorporated by reference, describes Doppler guide wires that can be used in the present invention, for example, bonded to the delivery sheath. In a preferred embodiment, the Doppler probe is not within the lumen of the delivery sheath.

In an embodiment of the methods and apparatuses described herein, the delivery sheath comprises only a single lumen. In an embodiment, the delivery sheath comprises only two lumens.

In an embodiment of the methods and apparatuses described herein, the delivery sheath comprises three or four lumens.

The lumens are internal and permit, for example, a pacing lead or ablation catheter placed therein to be advanced to the end of the lumen such that at least the tip of the lead or catheter is external to the lumen.

In an embodiment, the method does not use a temporary cardiac pacing lead. In an embodiment of the methods and apparatuses described herein, the delivery sheath does comprise a lumen containing a temporary cardiac pacing lead. In an embodiment, the method does not use a vacuum lumen. In an embodiment of the methods and apparatuses described herein, the delivery sheath does comprise a vacuum lumen.

In an embodiment of the methods and apparatuses described herein, the lumen of the delivery sheath is constructed such that both an epicardial pacing lead and an ablation catheter can be advanced within a lumen of the delivery sheath (but not simultaneously, and not present at the same time).

In an embodiment of the methods, the method is employed for left ventricular pacing. In an embodiment of the methods, the method is employed for ablation in treating ventricular tachycardia. In an embodiment of the methods, the method is employed for ablation in treating atrial fibrillation.

In an embodiment, the apparatus herein, and as used in the methods herein, does not comprise an intracardiac echo catheter.

In a preferred embodiment of the methods and apparatuses described herein, the blood vessel is a coronary blood vessel.

The methods and apparatuses described herein can be performed, or constructed, mutatis mutandis, with another suitable blood flow probe in place of the Doppler probe.

In an embodiment of the methods described herein, the methods further comprise passing a current through the epicardial pacing lead or ablation catheter and monitoring phrenic nerve activity of the subject, wherein phrenic nerve stimulation indicates that the location of the end of the sheath or catheter is not appropriate for epicardial pacing or is not appropriate for ablation.

In an embodiment of the methods described herein, the signal from the Doppler probe is an audio output is quantified by a monitoring device.

In an embodiment of the methods described herein, the methods further comprise assessing ventricular function of the heart when a pacing current is applied to the heart through the epicardial pacing lead.

In an embodiment of the methods described herein, ventricular function is assessed using an echocardiograph.

In an embodiment of the methods the arrhythmia originates on, or can be ablated from, the surface tissue of the heart.

In an embodiment of the methods, the delivery sheath is steerable delivery sheath. In an embodiment, it is bi-directionally steerable.

In an embodiment of the methods described herein regarding placing an epicardial pacing lead, the methods further comprise when the end of the sheath is determined to be within an unacceptable distance of any blood vessel of a predetermined size or above, (i) re-positioning the end of the sheath to a second predetermined position on the epicardium of the heart, spatially separate from the first predetermined position, and (ii) quantifying a signal obtained from the micro-Doppler crystal, so as to determine the proximity of the end of the sheath to a blood vessel of a predetermined size or above, wherein when no blood vessel of the predetermined size or above is determined to be within an unacceptable distance of the end of the sheath in the second predetermined position, advancing an epicardial pacing lead within the sheath to the end of the sheath so as to place the epicardial pacing lead onto the epicardium of the heart in the subject at the second predetermined position, and wherein when the end of the sheath in the second predetermined position is determined to be within an unacceptable distance of a blood vessel of a predetermined size or above, repeating steps (i) and (ii) until no blood vessel of the predetermined size or above is determined to be within an unacceptable distance of the end of the sheath blood vessel in a subsequent predetermined position, and when such a state is effected, advancing the epicardial pacing lead within lead delivery sheath to the end of the sheath so as to place the epicardial pacing lead onto the epicardium of the heart in the subject at the subsequent predetermined position.

In an embodiment of the methods described herein regarding placing an ablation catheter, the methods further comprise, when the end of the sheath is determined to be within an unacceptable distance of a blood vessel of a predetermined size or above, (i) re-positioning the end of the sheath to a second predetermined position on the epicardium of the heart, spatially separate from the first predetermined position, and (ii) quantifying a signal obtained from the micro-Doppler crystal, so as to determine the proximity of the end of the sheath to a blood vessel of a predetermined size or above, wherein when no blood vessel of the predetermined size or above is determined to be within an unacceptable distance to the end of the sheath in the second predetermined position, advancing an ablation catheter within the sheath to the end of the sheath so as to place the ablation catheter onto the epicardium of the heart in the subject at the second predetermined position, and wherein when the end of the sheath in the second predetermined position is determined to be within an unacceptable distance of a blood vessel of a predetermined size or above, repeating steps (i) and (ii) until no blood vessel of the predetermined size or above is determined to be within an unacceptable distance of the end of the sheath blood vessel in a subsequent predetermined position, and when such a state is effected, advancing the ablation catheter within lead delivery sheath to the end of the sheath so as to place the ablation catheter onto the epicardium of the heart in the subject at the subsequent predetermined position.

In an embodiment of the methods described herein, the predetermined position, second predetermined position and/or subsequent predetermined position is on the left ventricle of the heart.

In an embodiment of the methods described herein, the method further comprises withdrawing the delivery sheath from the subject after the epicardial pacing lead is placed or after the ablation catheter is placed.

In an embodiment of the methods described herein, the method is used for re-synchronization of the heart tissue.

Also provided is an apparatus comprising an epicardial pacing lead delivery sheath, or an ablation catheter delivery sheath, the delivery sheath comprising a lumen of a diameter sufficient for the advancement of an epicardial pacing lead or an ablation catheter therein, respectively, and comprising a Doppler blood-flow probe at the end of the delivery sheath.

In an embodiment of the apparatus, the Doppler blood-flow probe comprises a piezoelectric crystal. In an embodiment, the Doppler blood-flow probe comprises a microvascular Doppler probe. In an embodiment, the Doppler blood-flow probe comprises a 20 MHz microvascular Doppler probe. In an embodiment, the delivery sheath lumen is 2 F 3 F, 4 F, 5 F, 6 F, 7 F, 8 F, 9 F, 10 F, 11 F, 12 F, 13 F, 14 F, 15 F, 16 F, 17 F, 18 F, 19 F or 20 F in diameter, or of a diameter between any two of these values. In an embodiment, the epicardial pacing lead diameter is 1 F, 2 F, 3 F, 4 F, 5 F, 6 F, 7 F, 8 F or 9 F, or of a diameter between any two of these values. In an embodiment, the ablation catheter diameter is 1 F, 2 F 3 F, 4 F, 5 F, 6 F, 7 F, 8F or 9 F, or of a diameter between any two of these values. In an embodiment, the ablation catheter is a radiofrequency (RF) ablation catheter. In an embodiment, the delivery sheath is steerable delivery sheath. In an embodiment, the delivery sheath is steerable via controls at a handle of the sheath, which handle is at the distal end of the sheath relative to the end of the sheath having the Doppler blood-flow probe attached. In an embodiment, the delivery sheath is bidirectionally steerable. In an embodiment, the apparatus further comprises one or more epicardial pacing lead(s) or an ablation catheter, each with or without its own Doppler blood flow probe attached. The lumen diameter of the delivery sheath is larger then the diameter of ablation catheter or pacing lead. In an embodiment, the Doppler blood-flow probe is attached to the end of the delivery sheath as described herein.

In an embodiment of the methods or the apparatus, the epicardial pacing lead diameter is 1 F, 2 F, 3 F, 4 F, 5 F, 6 F, 7 F, 8F or 9 F, or of a diameter between any two of these values, for example 3.5 F, 4 F-5 F (such as 4.1 F), 5 F-6 F (such as 5.3 F, 5.7 F).

In an embodiment of the methods or the apparatus, the ablation catheter diameter is 1 F, 2 F, 3 F, 4 F, 5 F, 6 F, 7 F, 8F or 9 F, or of a diameter between any two of these values. In an embodiment, the ablation catheter is a radiofrequency (RF) ablation catheter. In an embodiment, the RF ablation catheter comprises 1, 2, 3 or 4 electrodes, or in excess of 4 electrodes. In an embodiment, the RF ablation catheter is curved. In an embodiment, the RF ablation catheter is steerable. In an embodiment, the ablation catheter is a cryoablation ablation catheter. In an embodiment, the cryoablation ablation catheter is capable of cryomapping, such that the user can determine the location of the arrhythmic source by reversibly cooling the tissue adjacent to the tip of the cryoablation catheter and monitoring the ensuing effect, if any, on the arrythmia. In an embodiment, the cryoablation ablation catheter is capable of cryoadhesion, such that the user can affix the tip of the catheter to the tissue adjacent to the tip.

In an embodiment of the methods or the apparatus, the delivery sheath diameter external diameter, or alternatively the lumen diameter, is 3 F, 4 F, 5 F, 6 F, 7 F, 8 F, 9 F, 10 F, 11 F, 12 F, 13 F, 14 F, 215 F, 16 F, 17 F, 18 F, 19 F or 20 F, or of a diameter between any two of these values. In a preferred embodiment, the delivery sheath is of greater diameter than the lead or catheter it permits delivery of.

The catheters of the invention may be steerable. In an embodiment, the catheters of the invention can be curved.

In an embodiment of the methods or the apparatus, the epicardial pacing lead comprises an attachment component, for example a screw mechanism, which allows the lead to be attached to the tissue of interest, e.g. cardiac tissue. In an embodiment, the epicardial pacing lead is extendable and retractable, which allows it to passively contact the tissue of interest. In an embodiment, the electrode of the lead comprises titanium nitride-coated platinum alloy, or is platinized (such as platinized platinum), or is titanium nitride coated. In an embodiment, the epicardial pacing lead is steroid-eluting. In an embodiment, the electrode of the epicardial pacing lead is unipolar. In an embodiment, the electrode of the epicardial pacing lead is bipolar. In an embodiment, the electrode of the epicardial pacing lead is quadripolar.

In an embodiment of the methods or the apparatus, the delivery sheath has an inner diameter of less than 6 F (for example, 5.5 F or 5.7 F) and an outer diameter of 6 F more than 6 F (for example 7 F or 8.4 F). In an embodiment, the ablation catheter sheath has an inner diameter of less than 6 F (for example, 5.5 F or 5.7 F) and an outer diameter of 6 F more than 6 F (for example 7 F or 8.4 F).

In an embodiment of the methods or the apparatus, the epicardial pacing lead is attached to an external pacemaker device (for example, see external pacemakers as sold by Medtronic Inc., Minneapolis, Minn., such as those providing 30-200 ppm, including continuously adjustable pacing).

Doppler blood flow probes that can be used in the present invention are well-known in the art. For example the tiny Doppler ultrasound probes presently used, for example those bonded within the lumen a needle (e.g., Doppler Smart Needle Technology, Smart Needle, Vascular Solutions Inc., Minneapolis, Minn. which uses a continuous wave Doppler) are used by physicians to access veins and arteries percutaneously, even though they are deep enough to be invisible from the surface of the body. Examples include crystal diode probes, piezoelectric crystal probes. Some probes are used to monitor post-operative vascular anastomoses, for example where probe is a crystal diode approximately 1 mm in diameter, affixed to a Silastic cuff (J. Reconstr Microsurg 2003; 19(5): 287-290). In an embodiment, the Doppler probe comprises a piezoelectric crystal and is attached to the tip or end of the catheter such that an ultrasonic beam can be effected to emit from the piezoelectric crystal into adjacent tissue including blood vessels. Reflected sound can be detected and emitted as audio or as some other sensory signal, for example visual.

Sheath-delivered pacemaker leads that can be used in the present invention are known in the art. Traditionally, epicardial pacemaker leads have been delivered either through a venous cut down or by percutaneous access to a vein in the shoulder region through which a short introducer sheath is placed. The epicardial pacing lead is then advanced through this short sheath which in that present invention comprises the Doppler blood flow probe, and guided to its target position in the heart by a shaped, removable stylet, which can be placed and removed from a central core channel in the pacemaker lead. More recently a permanent epicardial pacing leads have been developed, (e.g., Medtronic SelectSecure system, Medtronic, MN), that use a long steerable sheath, and a small (4 F) pacing lead with no central core stylet. When this lead is delivered to its target area and fixed using a tiny screw mechanism, the sheath is withdrawn, leaving in place a very small and flexible pacing lead.

Pericardial catheters that can be used in the present invention for ablation of cardiac tissue to treat arrhythmia are also well-known in the art. In this technique, in patients with arrhythmias originating on the surface of the heart, the pericardium is accessed using the subxyphoid approach with a long needle. After a guide wire is inserted into the pericardial space, a sheath, which in the present invention comprises a Doppler blood-flow probe, can be introduced over the wire using, for example, the traditional Seldinger technique. An ablating catheter can then be introduced through the sheath, and moved over the surface of the pericardium until the origin of the arrhythmia is identified. This abnormal area can then be destroyed by ablative techniques, for example, radiofrequency (RF) energy (for example, temperature controlled RF ablation or fluid-cooled RF ablation) or cryoenergy. In the art, with the current ablating catheter techniques, injections of the coronary artery are needed to assure that the ablation will not compromise an important nearby coronary artery. In some cases multiple coronary injections must be used. Thus, the risks of coronary angiography and radiographic contrast materials (x-ray dye) are added to those of the ablation procedure. The present invention, in contrast to the art (see Sosa et al. 2005), avoids this need for coronary angiography.

In an embodiment of the methods described herein, no coronary angiography is performed on the subject as part of the method. In an embodiment of the methods described herein, no coronary angiography is performed on the subject prior to placing the delivery sheath or needle. In an embodiment of the methods described herein, no coronary angiography is performed on the subject within 12 hours prior to placing the delivery sheath or needle. In an embodiment of the methods described herein, no coronary angiography is performed on the subject within 24 hours prior to placing the delivery sheath or needle. In an embodiment of the methods described herein, no coronary angiography is performed on the subject within 48 hours prior to placing the delivery sheath or needle.

The present invention, in a non-limiting embodiment, comprises or comprises use of, a Doppler blood flow probe attached to the tip, or an end, of an epicardial pacing lead delivery sheath, or to the tip, or an end, of a steerable ablation catheter sheath, such that the blood flow proximal to the Doppler blood flow probe can be determined, quantified, and/or monitored when inserted into a subject's body so as to permit detection of blood vessels in the cardiac or pericardial area close to the Doppler blood flow probe and therefore the area close to the tip or end of a epicardial pacing lead delivery sheath, or to the steerable ablation catheter sheath. Accordingly, the Doppler probe is attached to the tip or end of the catheter such that the signal from the Doppler probe can be received by an appropriately attached monitor external to the subject's body. In an embodiment, the attached Doppler probe is within the lumen of the end portion of the delivery sheath. In a preferred embodiment, the Doppler probe is in an external surface of the delivery sheath, i.e. not in the lumen, for example at the external tip of the sheath or on a surface of the sheath which surface is external to the lumen.

In an embodiment of the methods or the apparatus described herein wherein the Doppler blood-flow probe is within 0.1, 0.2, 0.3, 0.4, 0.5, 0.75, 1, 2, 3, 4, 5, 6, 7, 8, 9, or 10 mm of the tip of the delivery sheath (either externally positioned on the sheath or internally, i.e in the lumen). In an embodiment of the methods or the apparatus described herein wherein the Doppler blood-flow probe is on the ablation catheter, the probe is within 0.5, 1, 2, 3, 4, 5, 6, 7, 8, 9, or 10 mm of the tip of the ablation catheter. In an embodiment of the methods or the apparatus described herein wherein the Doppler blood-flow probe is on the epicardial pacing lead, the probe is within 0.5, 1, 2, 3, 4, 5, 6, 7, 8, 9, or 10 mm of the tip of the ablation catheter.

The signal form the Doppler blood-flow probe of the methods or apparatuses may be an electromagnetic signal. In an embodiment, the signal from the Doppler blood-flow probe of the methods or apparatuses may be light. In an embodiment, the signal from the Doppler blood-flow probe of the methods or apparatuses may be electric. In an embodiment, the signal from the Doppler blood-flow probe of the methods or apparatuses may be audio.

In an embodiment of the methods described herein, the epicardial pacing leads or ablation catheter are placed without the subject undergoing thoractomy. In an embodiment of the methods described herein, the epicardial pacing leads or ablation catheter are placed without being placed through a percutaneous venous route.

In an embodiment of the methods described herein, the predetermined size of the blood vessel is 1.0 mm, 1.05 mm, 1.1 mm, 1.15 mm, 1.2 mm, 1.25 mm, 1.3 mm, 1.35 mm, 1.4 mm, 1.45 mm, 1.5 mm, 1.55 mm, 1.6 mm, 1.65 mm, 1.7 mm, 1.75 mm, 1.8 mm, 1.85 mm, 1.9 mm, 1.95 mm, 2.0 mm, 2.05 mm, 2.1 mm, 2.15 mm, 2.2 mm, 2.25 mm, 2.3 mm, 2.35 mm, 2.4 mm, 2.45 mm, 2.5 mm, 2.55 mm, 2.6 mm, 2.65 mm, 2.7 mm, 2.75 mm, 2.8 mm, 2.85 mm, 2.9 mm, 2.95 mm, 3.0 mm or above, or is of a size between any two of these values. In an embodiment, the predetermined size of the blood vessel is 1.8-2.2 mm or above.

The distance between the tip of the sheath, lead, needle or catheter and the blood vessels of the predetermined size which is considered an unacceptable distance (e.g., for the purposes of performing the procedure, so as to recommend against that position being a usable pacing site for example) is readily determined by one skilled in the art. Determinations based on proximity can be made by the user of the method, for example, a cardiologist. In an embodiment, within 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1.0 mm, 1.05 mm, 1.1 mm, 1.15 mm, 1.2 mm, 1.25 mm, 1.3 mm, 1.35 mm, 1.4 mm, 1.45 mm, 1.5 mm, 1.55 mm, 1.6 mm, 1.65 mm, 1.7 mm, 1.75 mm, 1.8 mm, 1.85 mm, 1.9 mm, 1.95 mm, 2.0 mm, 2.05 mm, 2.1 mm, 2.15 mm, 2.2 mm, 2.25 mm, 2.3 mm, 2.35 mm, 2.4 mm, 2.45 mm, 2.5 mm, 2.55 mm,2.6 mm, 2.65 mm, 2.7 mm, 2.75 mm, 2.8 mm, 2.85 mm, 2.9 mm, 2.95 mm, or 3.0 mm or above is considered an unacceptable distance to the blood vessel of the predetermined size. In an embodiment, any range or sub-range within these distances is considered an unacceptable distance to the blood vessel of the predetermined size. In an embodiment, the unacceptable distance is a predetermined distance.

In an embodiment of the methods and/or apparatuses described herein, the lead, sheath or catheter further comprises a fiberoptic sensor attached thereto which permits the user visualization of the tip or end of the lead, sheath or catheter, and/or visualization of the tissue immediately in front of the tip of the lead, sheath or catheter.

The subject of the methods may be any subject. Preferably, the subject is a mammal. More preferably, the subject is a human.

Also provided is a system comprising the apparatuses described herein and a monitor attachable to or attached to the apparatus which monitor displays or emits a signal obtained from the Doppler blood-flow probe. In an embodiment, the system further comprises a computer (1) having a display device for displaying information obtained from the Doppler blood-flow probe and/or (2) for controlling electrical current to the epicardial pacing lead(s) and/or energy to the ablation catheter. Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions for the methods of the invention encoded on a computer readable medium for execution by, or to control the operation of, data processing apparatus. The computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes described in this specification can be performed with employing one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., in non-limiting examples, a CRT (cathode ray tube) or LCD (liquid crystal display) monitor, for displaying information (e.g. visual or blood flow signals) to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback (especially useful for Doppler blood flow probes), or tactile feedback; and input from the user can be received in any form, including acoustic, speech, or tactile input, delivery sheath steering controls, ablation catheter controls, epicardial pacing lead controls.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

As used herein “and/or”, for example as in option A and/or option B, means the following embodiments: (i) option A, (ii) option B, and (iii) the option A plus B, and any subset of such options, including only one option.

All combinations of the various elements described herein are within the scope of the invention unless otherwise indicated herein or otherwise clearly contradicted by context.

Where a numerical range is provided herein, it is understood that all numerical subsets of that range, and all the numerical values to one decimal place, are provided as part of the invention. Thus, for example, an ablation catheter which is 3 F-4 F in diameter includes the subset of ablation catheters which are 3.1 F, 3.2 F, 3.3 F etc. in diameter as well as the range of ablation catheters which are 3.3 F to 3.6 F, 3.1 F to 4 F and so forth.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### EXAMPLES

In an example, a micro-Doppler crystal (such as that used with the Smart Needle Doppler described above) is bonded externally to the tip of (e.g.) a Medtronic SelectSecure lead delivery sheath for pacing purposes, or of a compatible steerable delivery sheath such as those used for ablation. The sheath is then advanced, e.g. using a subxyphoid approach, into the pericardial space. In the case of pacing, when the tip of the sheath reaches an area of the external surface of the LV that is deemed as appearing appropriate for left ventricular pacing, the Doppler probe is used to determine that no blood vessel is in close proximity which blood vessel, in the estimation of the user (for example a physician) is of a size that would be deleterious to the subject's health if adversely affected by the procedure. The lead can then be extended and tested with, or without, fixation to the myocardium. If desired, echocardiographic or other measurements can be made to assess effects on ventricular function.

If a satisfactory site is identified, e.g. one that does not produce phrenic nerve stimulation and which is not in close proximity to a large blood vessel as assessed by the Doppler probe, the steerable sheath can be angled towards the myocardium and the lead fixed in place using, for example, a standard screw mechanism. Alternatively, the lead can be passively contacted to the myocardium. Once satisfactory signals and pacing are confirmed, the sheath is withdrawn from the pericardium, leaving the pacemaker lead on the epicardial surface of the left ventricle. From the subxyphoid position, the lead can then be tunneled up to the pacemaker pocket or ICD pocket using a commercially available tunneling tool (e.g. Traverser Pacemaker Lead Tunneling Tool, Pressure Products, Inc.).

For ablation, an analogous process can be used with a steerable sheath comprising an attached Doppler blood flow probe at the tip or end thereof, to deliver a mapping and ablation lead/catheter to a targeted area. The Doppler probe signal can confirm presence or absence of nearby vessels before ablation is initiated without the need for coronary angiography. The mapping can be used prior to ablation to determine the arrhythmia source.

The advantages of the present invention are various and include LV lead placement without regard to coronary sinus anatomy, left ventricular lead placement without need for thoracotomy, choice of left ventricular sites for optimal CRT, while avoiding phrenic nerve stimulation and large epicardial vessels, no need for coronary angiography during pacer lead placement, and avoidance of repetitive coronary injections during ablation procedures.

In addition, there is no need for special operating rooms or equipment beyond those commonly available in electrophysiology laboratories that perform device implants.

### REFERENCES

1. J. Reconstr Microsurg 2003; 19(5): 287-290.
2. Sosa et al., J. Cardiovasc. Electrophysiol. 16:449-452 (2005).

1. A method for placing an epicardial pacing lead or an ablation catheter onto the epicardium of a heart in a subject, comprising placing an end of an epicardial pacing lead delivery sheath, or an ablation catheter delivery sheath, the delivery sheath comprising a lumen and comprising a Doppler blood-flow probe at the end of the delivery sheath, at a predetermined position adjacent to the epicardium of the heart, obtaining a signal from the Doppler blood-flow probe so as to determine the proximity of the end of the sheath to a blood vessel of a predetermined size or above and, when no blood vessel of the predetermined size or above is determined to be within an unacceptable distance of the end of the sheath, advancing an epicardial pacing lead, or an ablation catheter, respectively, within the lumen of the delivery sheath to the end of the sheath so as to place the epicardial pacing lead, or the ablation catheter, respectively, onto the epicardium of the heart in the subject.

2. The method of claim 1, comprising placing the epicardial pacing lead, and further comprising administering to the subject an electrical current through the epicardial pacing lead, and through a second epicardial pacing lead positioned in a right ventricle of, or right ventricle portion of a septal wall of, the heart of the subject, so as to deliver a synchronizing electrical current to the heart of the subject.

3. A method for placing an ablation catheter onto the epicardium of a heart in a subject, comprising placing an end of an ablation catheter delivery sheath, the sheath comprising a lumen, at a predetermined position adjacent to the epicardium of the heart, advancing an ablation catheter comprising a Doppler blood-flow probe at an end thereof within the lumen of the sheath to the end of the sheath proximal to the epicardium, and obtaining a signal from the Doppler blood-flow probe so as to determine the proximity of the end of the ablation catheter to a blood vessel of a predetermined size or above and, when no blood vessel of the predetermined size or above is determined to be within an unacceptable distance of the end of the sheath, placing the ablation catheter comprising the Doppler blood-flow probe onto the epicardium of the heart in the subject.

4. The method of claim 1, wherein the ablation catheter is placed onto the epicardium of the heart, further comprising administering to the subject an electrical current or a radiofrequency energy or a cryogenic material through the ablation catheter to the epicardium in an amount effective to ablate a portion of the cardiac tissue of the heart.

5. (canceled)

6. The method of claim 1, wherein the subject has an arrhythmia.

7. The method of claim 6, wherein the arrhythmia comprises one or more of sinus node dysfunction, junctional ectopic tachycardia, a supraventricular tachycardia or atrioventricular block, and/or is a post-operative arrhythmia.

8. The method of claim 6, wherein the arrhythmia comprises a supraventricular tachycardia and is an atrio-ventrical nodal reentry, atrio-ventricular reentry, atrial flutter or sinus node reentry tachycardia.

9. The method of claim 1, wherein the subject has had cardiac surgery.

10. The method of claim 1, wherein the epicardial pacing lead delivery sheath, or the ablation catheter delivery sheath, is introduced into the subject via a subxyphoid route.

11-12. (canceled)

13. The method of claim 1, further comprising passing a current through the epicardial pacing lead or ablation catheter and monitoring phrenic nerve activity of the subject, wherein phrenic nerve stimulation indicates that the location of the end of the sheath is not appropriate for epicardial pacing or is not appropriate for ablation.

14. (canceled)

15. The method of claim 1, further comprising assessing ventricular function of the heart when a pacing current is applied to the heart through the epicardial pacing lead.

16. The method of claim 15, wherein ventricular function is assessed using an echocardiograph.

17. The method of claim 1, wherein the epicardial pacing lead is placed, and further comprising, if the end of the sheath is determined to be within an unacceptable distance of a blood vessel of a predetermined size or above, (i) re-positioning the end of the sheath to a second predetermined position on the epicardium of the heart, spatially separate from the first predetermined position, and (ii) quantifying a signal obtained from the Doppler blood-flow probe, so as to determine the proximity of the end of the sheath to a blood vessel of a predetermined size or above, wherein when no blood vessel of the predetermined size or above is determined to be within an unacceptable distance of the end of the sheath in the second predetermined position, advancing an epicardial pacing lead within the sheath to the end of the sheath so as to place the epicardial pacing lead onto the epicardium of the heart in the subject at the second predetermined position, and wherein when the end of the sheath in the second predetermined position is determined to be within an unacceptable distance of a blood vessel of a predetermined size or above, repeating steps (i) and (ii) until no blood vessel of the predetermined size or above is determined to be within an unacceptable distance of the end of the sheath blood vessel in a subsequent predetermined position, and when such a state is effected, advancing the epicardial pacing lead within lead delivery sheath to the end of the sheath so as to place the epicardial pacing lead onto the epicardium of the heart in the subject at the subsequent predetermined position.

18. The method of claim 1, wherein the ablation catheter is placed, and further comprising, if the end of the sheath is determined to be within an unacceptable distance of a blood vessel of a predetermined size or above, (i) re-positioning the end of the sheath to a second predetermined position on the epicardium of the heart, spatially separate from the first predetermined position, and (ii) quantifying a signal obtained from the Doppler blood-flow probe, so as to determine the proximity of the end of the sheath to a blood vessel of a predetermined size or above, wherein when no blood vessel of the predetermined size or above is determined to be within an unacceptable distance of the end of the sheath in the second predetermined position, advancing an ablation catheter within the sheath to the end of the sheath so as to place the ablation catheter onto the epicardium of the heart in the subject at the second predetermined position, and wherein when the end of the sheath in the second predetermined position is determined to be within an unacceptable distance of a blood vessel of a predetermined size or above, repeating steps (i) and (ii) until no blood vessel of the predetermined size or above is determined to be within an unacceptable distance of the end of the sheath blood vessel in a subsequent predetermined position, and when such a state is effected, advancing the ablation catheter within lead delivery sheath to the end of the sheath so as to place the ablation catheter onto the epicardium of the heart in the subject at the subsequent predetermined position.

19-21. (canceled)

22. An apparatus comprising an epicardial pacing lead delivery sheath, or an ablation catheter delivery sheath, the delivery sheath comprising a lumen of a diameter sufficient for the advancement of an epicardial pacing lead or an ablation catheter therein, respectively, and comprising a Doppler blood-flow probe at the end of the delivery sheath.

23. The apparatus of claim 22, wherein the Doppler blood-flow probe comprises a piezoelectric crystal.

24. The apparatus of claim 22, wherein the Doppler blood-flow probe comprises a 20 MHz microvascular Doppler probe.

25. The apparatus of claim 22, wherein the delivery sheath lumen is 3 F, 4 F, 5 F, 6 F, 7 F, 8 F, 9 F, 10 F, 11 F, 12 F, 13 F, 14 F, 15 F, 16 F, 17 F, 18 F, 19 F or 20 F in diameter, or of a diameter between any two of these values.

26-27. (canceled)

28. The apparatus of claim 22, wherein the ablation catheter is a radiofrequency (RF) ablation catheter.

29. The apparatus of claim 22, wherein the delivery sheath is steerable delivery sheath.

30-34. (canceled)
